(19) United States
(12) Patent Application Publication
(10) Pub. No.: US 2002/0119502 A1
(43) Pub. Date: Aug. 29, 2002

(54) Combination therapy for the treatment of estrogen-sensitive disease

(76) Inventors: Mark Stanford Moran, Orinda, CA (US); Peter J. Langecker, Monte Sereno, CA (US)

Correspondence Address: COOLEY GODWARD, LLP, 3000 EL CAMINO REAL, 5 PALO ALTO SQUARE, PALO ALTO, CA, 94306, US

(21) Appl. No.: 09/970,509

(22) Filed: Oct. 3, 2001

Related U.S. Application Data
(60) Provisional application No. 60/238,772, filed on Oct. 6, 2000.

(51) Int. Cl. A61K 31/56; A61K 31/137; G01N 33/574

(52) U.S. Cl. 435/7.23; 514/171; 514/651

(57) ABSTRACT

The present invention provides methods which increase the effectiveness of combination drug therapies for treating estrogen-sensitive diseases, such as breast cancer, as well as novel combinations useful to treat such diseases.

### CROSS-REFERENCE TO RELATED APPLICATIONS

This application claims the benefit of priority under 35 U.S.C. § 119(e) to application Ser. No. 60/238,772, filed Oct. 6, 2000, the contents of which are incorporated herein by reference.

### FIELD OF THE INVENTION

This invention relates to a method of allowing or increasing the effectiveness of combination drug therapy for treating an estrogen-sensitive disease, such as breast cancer. The invention further relates to novel combination drug therapies and methods for treating an estrogen-sensitive disease.

### BACKGROUND OF THE INVENTION

U.S. Pat. No. 5,550,107 to Fernand Labrie and others teach certain combination drug therapies for the treatment of estrogen-sensitive disease, e.g., breast and endometrial cancer. The patent teaches the combination of any of the listed antiestrogen drugs with any of the other listed drugs including androgen, a progestin, or an inhibitor of sex steroid formation, ACTH secretion, prolactin secretion, or growth hormone secretion.

One overarching problem with such otherwise unspecified combination treatments for breast cancer identified in the Labrie patent is the potential for one drug, or class of drugs, to cause an adverse effect on the absorption, distribution, excretion, or metabolism (or some combination of these) of one of the other drugs, or other classes of drugs, employed in the combination regimen. This problem is especially noteworthy in postmenopausal women who have primary or recurrent metastatic disease when surgery or radiation therapy may no longer be feasible and pharmaceutical therapy is the principal remaining treatment option. This kind of problem has been highlighted clinically in the setting of partially or completely failed attempts to combine an antiestrogen with an inhibitor of sex steroid hormone synthesis such as an aromatase inhibitor. Recent preclinical and clinical studies have shown that it is not possible to select any antiestrogen and pair it with any aromatase inhibitor to achieve the desired effect. Such unguided pairings lead to treatment outcomes that are less than expected, with lesser antitumor effects or potentially even tumor stimulation, and that, as a result, may prove to be harmful to the patient.

It still remains desirable to control detrimental hormones (i.e., those hormones which may stimulate tumor growth) and to potentially block multiple pathways by which these hormones may be made in the body. However, prior art combination treatments have ignored the possibility that a drug that may, when used as monotherapy, block one hormone pathway may no longer do so as effectively, if at all, when another (interfering) drug is also present in the body. This unrecognized problem has the effect of leaving pathways wholly or partially available for formation of undesired tumor-stimulating hormones, thus diminishing or negating the desired beneficial effect of the combination.

When considering antiestrogens as part of a combination regimen for treating breast cancer, prior art has emphasized the importance of selecting a “pure” estrogen antagonist having relatively little intrinsic estrogen agonist activity to the exclusion of other potentially important factors. If the purity of the antiestrogen activity were the only factor that were important in the selection of an antiestrogen as part of a combination regimen, then selection based on purity of activity would yield uniformly predictable and beneficial clinical results. This is demonstrably untrue. Even a pure antiestrogen, however, can interact adversely with other drugs. Similar deficiencies of reasoning have characterized prior recommendations regarding the selection of, for example, an inhibitor of sex steroid hormone synthesis such as an aromatase inhibitor. Prior art focuses on purity and selectivity to the relative exclusion of other essential factors in the selection process, namely, co-selection of the other drug(s) in the regimen, and the potential for adverse effects of one drug on the absorption, distribution, metabolism, or excretion of any other drug in the regimen.

In the setting of the combination of an antiestrogen with an aromatase inhibitor, for example, prior art recommendations for establishing dosing regimens have ignored the potential that one drug in a combination may adversely influence the absorption of a second or third drug in the combination, leading either to low plasma levels with concomitantly reduced efficacy or unexpectedly high plasma levels that increase the risk of toxicity. For example, the Labrie U.S. Pat. No. 5,550,107 gives suggested dose ranges for the drugs to be combined, but says nothing about possible adverse interactions, their potential effect on the target dose ranges, how to achieve a target range in the setting of one or more adverse interactions, and (most importantly) how to avoid such adverse interactions in the first place. See columns 22-24 of Labrie.

Prior art has also ignored the potential that one drug in a combination may adversely influence the distribution of a second or third drug in the combination. Distributional changes can occur because of changes in compartmentalization within the body, changes in transfer rates between compartments, changes in binding of one or more drugs to proteins that may serve as circulating “depots”, or because of a combination of these factors. If the distribution is altered, then one or more drugs may not reach the tumor in adequate concentrations or intratumoral concentrations may be diminished. Such effects would reduce the efficacy and potentially the safety of the combination. The Labrie patent does not address these important factors that may determine the utility and safety of a combination regimen.

Moreover, although prior art has, in general terms, warned of possible adverse effects of one drug on the metabolism of another, prior art has consistently failed to provide guidance on how best to avoid or to mitigate such interferences. Labrie also fails to provide teaching with respect to this adverse interaction. Such changes in metabolism can be the result of effects on one or more organs or tissues involved in disposal of one of the drugs in the pair.

Finally, in this setting of antiestrogen-aromatase inhibitor pairing, prior art recommendations regarding dosing regimens have fundamentally ignored the potential for drug-drug interactions on excretory pathways. Changes in excretion can also lead to changes in plasma or tissue levels that can adversely impact safety or effectiveness or both. Prior art has neglected the potential for multiple interactions affecting simultaneously two or more factors from absorption, distribution, excretion and metabolism. Finally, the extent of interference may change with timing of dosing and the passage of time, effects ignored by prior art, particularly in the setting of combination therapy involving an antiestrogen and an aromatase inhibitor.

In addition, prior art also has tended to emphasize the (sole) utility of measuring plasma levels of estrogens to assess effectiveness of treatment. However, plasma levels of estrogens are at best a surrogate for the level of estrogens present at the estrogen receptor(s) and plasma levels may be less than, substantially the same as, or greater than levels within a tumor itself. Moreover, endogenous estrogen levels may rise with the use of an antiestrogen that blocks estrogen receptors (causing feedback stimulation of estrogen production) while estrogen levels may decline with the use of an aromatase inhibitor. When both classes of drugs are utilized together, the actual interpretation of plasma levels becomes uncertain. This difficulty in interpretation is made significantly greater when a regimen is utilized in which one drug interferes with the activity of another, and particularly when that interference changes with time. Moreover, the sensitivity of a tumor cell to an estrogen or an estrogen analogue can also change with time so that progressive falls in plasma levels of estrogens can still be associated with marked tumor stimulatory effects.

We have now discovered that combination drug treatment of estrogen-sensitive diseases can be made possible or significantly improved by considering the full spectrum of characteristics of each drug in the regimen before administering the combination and not just purity of activity or relative receptor selectivity. These characteristics include: the absorption, distribution, metabolism, and excretion of each drug in the regimen and the potential effect on the other drug (or drugs), and possibly certain endocrinological effects. The difficulties in interpreting only biochemical markers of “antiestrogen therapy,” such as plasma markers, serve to emphasize the importance of properly choosing regimens so that unwanted changes in absorption, distribution, metabolism, and/or excretion are minimized or eliminated.

The deficiencies of the prior art necessarily diminish the effectiveness of treatment, increase the cost of effective treatment, increase the risk of toxic side effects, and increase the cost of treating such side effects.

### SUMMARY OF THE INVENTION

One aspect of this invention is a process for identifying a combination of pharmacological agents for the prevention or treatment of breast cancer in an animal, preferably a human female. The process comprises

identifying antiestrogen drugs and a therapeutically-effective dosage range for an antiestrogen so identified;

determining the relevant aspects of absorption, distribution, metabolism, and excretion (ADME) characteristics for the antiestrogen;

identifying sex steroid enzyme inhibitor drugs and therapeutically-effective dosage ranges for an enzyme inhibitors so identified;

determining the relevant aspects of ADME characteristics of an enzyme inhibitor so identified;

choosing each drug and a dosage range for each drug such that each drug exhibits useful therapeutic activity but each drug exhibits minimal interference with ADME of the other drug.

Another aspect of this invention is a method of treating breast cancer in patient or preventing breast cancer in an animal (preferably a human female) predisposed to breast cancer. The process comprises administering to the animal a therapeutically-effective amount of an antiestrogen drug and concurrently administering a therapeutically-effective amount of a sex steroid enzyme inhibitor drug, wherein the antiestrogen and the enzyme inhibitor, and dosage ranges for each, are chosen so that there is minimal material interference with the absorption, distribution, metabolism, and excretion (ADME) of the other drug.

Another aspect of this invention is a process for optimizing treatment of a breast cancer patient or for optimizing a cancer-preventive regimen for an animal predisposed to such cancer. The process comprises

identifying antiestrogens and a therapeutically-effective dosage range for the antiestrogen so identified;

determining the relevant aspects of ADME characteristics for the antiestrogen in the patient;

identifying sex steroid enzyme inhibitors and a therapeutically-effective dosage range for an enzyme inhibitor so identified;

determining the relevant aspects of ADME characteristics for the enzyme inhibitor so identified;

selecting the antiestrogen and an enzyme inhibitor, and a dosage range for each, so that material interference by one drug on the other drug is minimized with respect to the ADME characteristics of one towards the other; and

co-administering the selected antiestrogen and enzyme inhibitor to the patient at the appropriate dosages.

Another aspect of this invention is a kit useful for treating breast cancer in a patient in need of treatment or for preventing breast cancer in a patient predisposed to cancer. The kit comprises an antiestrogen drug in a dosage form to provide a therapeutically effective amount of the antiestrogen and a therapeutically effective amount of an sex steroid enzyme inhibitor drug, wherein the dosage form and amount of each drug are chosen so that material interference is minimized with respect to ADME characteristics of one drug towards the other drug.

Still another aspect of the invention is a pharmaceutical composition for treating or preventing breast cancer. The composition comprises a therapeutically-effective amount of an antiestrogen agent and a therapeutically-effective amount of a sex steroid enzyme inhibitor drug wherein the amount of each drug is chosen so that there is minimal material interference between one drug's ADME characteristics and the other drug's ADME characteristics in a patient.

Other aspects will be apparent upon reading the detailed description of the invention.

### BRIEF DESCRIPTION OF THE FIGURE

FIG. 1 is a schematic diagram of some of the sites of action of the entities that may play a role in the production of steroids in a mammal.

### DETAILED DESCRIPTION AND PRESENTLY PREFERRED EMBODIMENT.

This invention is based in part on the discovery of how to identify certain effective combination regimens or how to maximize the effectiveness of other combination regimens for the treatment or prevention of cancer, particularly breast cancer, via avoiding or minimizing certain drug-drug interactions. In the past it has been broadly taught that breast and endometrial cancer can be treated by administering any antiestrogen and any other hormonal agent, such as an androgenic agent, a progestin, or inhibitor of an enzyme that catalyzes a step in the synthesis a sex steroids from their precursors in peripheral tissues through a non-adrenal mechanism (see U.S. Pat. No. 5,550,107 to Labrie). Other agents also can be used. Random combinations are unlikely to yield useful regimens for the reasons stated in the “Background of the Invention,” and, accordingly, the development of combinations and dosages that are therapeutically effective has proven elusive. We believe that the prevention or treatment of a breast cancer in a patient, either male or female, can be made possible or optimized by taking into account certain interactions between the antiestrogen and the inhibitor (and other agents) that are part of the combination.

“Treatment” of breast cancer means the administration of the combination therapy of this invention to reduce the presence of the cancer (e.g., reduce tumor size), to prevent the expansion of the cancer (e.g., prevent the cancer from spreading), or to stabilize the cancer.

“Prevention” of breast cancer means the administration of the combination therapy of this invention to prevent the onset or recurrence of a cancerous condition in a subject predisposed to, or at risk of, the condition.

One aspect of the invention can be viewed as a process for identifying a combination treatment of breast cancer in a warm-blooded animal. While the invention is useful in any warm-blooded animal that may develop breast cancer, it is most useful for human female patients, as the vast majority of cases treated are in human females. The process comprises identifying a antiestrogen and a therapeutically-effective dosage range for the antiestrogen, i.e., a dosage range showing anticancer activity. The relevant aspects of absorption, distribution, metabolism, and excretion characteristics of the antiestrogen in the patient are determined. At least one other agent having tumor-inhibiting activity, i.e., an inhibitor of sex steroid biosynthesis, is identified along with a dosage range for the other agent. The degree of interference of each agent with the other agent's absorption, distribution, metabolism, and excretion is determined, and the dosage of the two agents chosen for combination is adjusted to maximize the therapeutically-effective anti-tumor activity of the two agents while minimizing material interference on the absorption, distribution, metabolism, or excretion function of the agents. Preferably, the antiestrogen and the other agent are chosen so that there is no material interference with the absorption, distribution, metabolism, and excretion (ADME) for either agent. The combination may also include an androgen, a progestin, a glucocorticoid, or an inhibitor of growth hormone secretion, prolactin secretion, or ACTH secretion, also chosen so that there is minimal material interference with the ADME characteristics.

In conjunction with teaching of how to make and use the invention, it is useful to discuss the presently understood background behind breast cancer treatment using tumor-inhibitory compounds, i.e., compounds showing anticancer activity.

Many breast cancer tumors are estrogen dependent, i.e., the growth of the tumor is aided by the presence of estrogen, which activates a receptor that sends a signal for the tumor cells to multiply. In the presence of an antiestrogen, a tumor will not grow as fast, may not grow at all or may shrink in size. An antiestrogen is a substance capable of preventing full expression of the biological effects of estrogenic hormones on responsive tissues e.g., by competing with estrogens at estrogen receptors at the cellular level and blocking the receptor. Such an “antiestrogen” is a compound that blocks an estrogen receptor in the target cell and can be referred to as an estrogen receptor blocker or ERB. However, estrogen may still be manufactured in the patient's tissues and some of that estrogen may find receptors and still send a growth signal. Thus, the idea of using another tumor-inhibitory agent, e.g., an inhibitor of an enzyme that catalyzes the production of a sex steroid that could fit into an estrogen receptor, arises (e.g., see U.S. Pat. No. 5,550,107). As mentioned previously, the problem we have identified is that the antiestrogen and the other agent will or can interfere with the other's absorption, distribution, metabolism and excretion characteristics and thus the effectiveness of the individual agents in combination is adversely affected. Our solution to the problem gives a Non-interfering Combination that retains Effectiveness, abbreviated as a “NCE” composition.

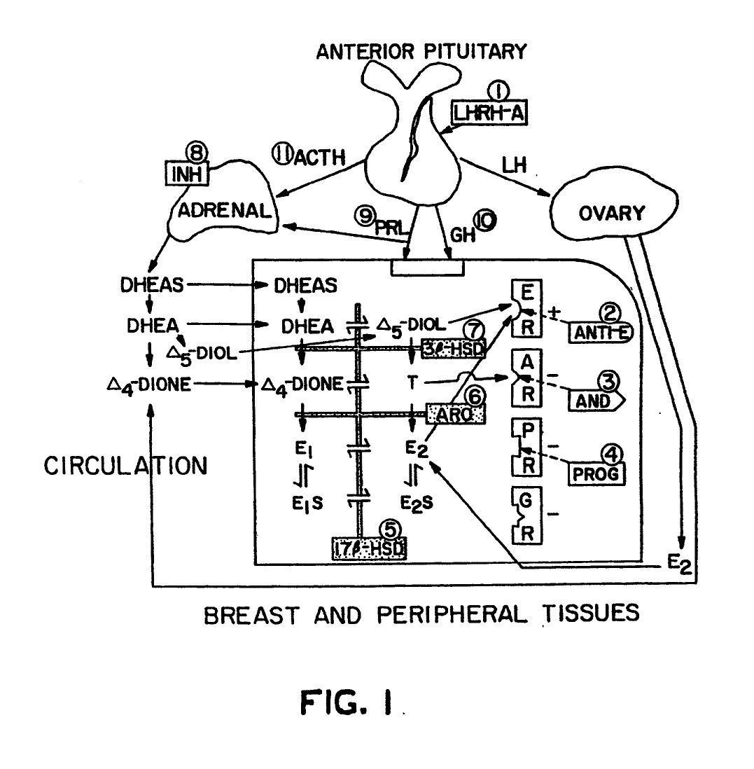

FIG. 1 is a schematic diagram of some of the sites of action of the entities that may play a role in the production of steroids in a mammal.

The following abbreviations are used in FIG. 1:

| | |
|---|---|
| ER: | estrogen receptor |
| AR: | androgen receptor |
| PR: | progesterone receptor |
| GR: | glucocorticoid receptor |
| DHEAS: | dehydroepiandrosterone sulfate |
| DHEA: | dehydroepiandrosterone |
| DELTA.5-diol: | androst-5-ene-3β,17β-diol |
| DELTA.4-dione: | androstenedione |
| E: | estrogen |
| E1: | estrone |
| E2: | 17β-estradiol |
| T: | testosterone |
| DHT: | dihydrotestosterone |
| E2 S: | E2 -sulfate |
| E1 S | E1 sulfate |
| (1) LHRH-A | luteinizing hormone-releasing hormone |
| | agonist or antagonist; |
| (2) ANTI-E: | antiestrogen |
| (3) AND: | androgen |
| (4) PROG: | progestin |
| (5) 17β-HSD | inhibitor of 17β-estradiol steroid dehydrogenase or |
| | 17β-hydroxysteroid dehydrogenase |
| (6) ARO: | inhibitor of aromatase activity |
| (7) 3β-HSD: | inhibitor of 3β-hydroxysteroid, |
| | .DELTA.5 -.DELTA.4 isomerase; |
| (8) INH: | inhibitor of adrenal steroidogenesis |
| (9) IPRL: | inhibitor of prolactin secretion |
| (10) IGH: | inhibitor of growth hormone secretion |
| (11) IACTH: | inhibitor of ACTH secretion |
| (12) NCE: | Non-Interfering Combination that retains Effectiveness |
| (13) LTED: | Long term estrogen deprivation |

Referring to FIG. 1, the “+”s and “−”s next to each indicated receptor designate whether activation of that receptor aids or hinders tumor growth. As may be seen from FIG. 1, activation of the estrogen receptor will stimulate tumor growth and is therefore to be prevented. However, it is important to continue to activate other receptors, whose activation may inhibit tumor growth, e.g., the androgen receptor, the progesterone receptor, and the glucocorticoid receptor.

Estrogen may be produced by a number of pathways, all of which can be affected by the pituitary. (1) luteinizing hormone (LH) stimulates the ovaries to release E2, which is then metabolized to estrogen with the aid of aromatase; (2) LH stimulates the ovaries to release DELTA 4-dione, which is then metabolized to El and on to estrogen; (3) andrenocorticotropic hormone (ACTH) is secreted to stimulate the adrenal glands to produce DHEAS, which can be metabolized to estrogen; (4) prolactin secretion may stimulate the adrenals to produce DHEAS and on to estrogen. As can be seen, there are various routes to block the production of estrogen to minimize the amount available for the estrogen receptor.

One method of inhibiting activation of the estrogen receptor is treatment with an effective and properly selected antiestrogen compound, suitable for inclusion in an NCE regimen, and having an affinity for the receptor site such that it binds the receptor site and blocks estrogen from binding and activating the site. It can be useful to select antiestrogens which tend to be pure antagonists, and which have no agonistic activity, but only if the selection retains the benefits of establishing an NCE regimen. Otherwise, the purity and relative absence of estrogen agonistic activity may be overcome by other factors such as changes in ADME characteristics or endocrine characteristics, of one or more concurrently administered drugs, as discussed hereinafter. Examples of antiestrogens are discussed in detail hereinafter.

Because it is extremely difficult to block all receptor sites, it is desirable to simultaneously decrease the concentration of estrogen available to activate estrogen receptors, i.e., to reduce the ovarian hormonal secretions. Hence, it is desirable to inhibit production of estrogen by the ovaries. In postmenopausal women, ovarian function has effectively ceased and no antiovarian therapy is generally required as such. However, in pre-menopausal women, it may be useful to suppress ovarian function either by chemical or surgical means. Chemically this can be accomplished by administering an appropriate luteinizing hormone releasing hormone (“LHRH” also referred to as GnRH or gonad releasing hormone) agonist or antagonist, such as those discussed hereinafter. Surgically, this can be accomplished by removing the ovaries through an oophorectomy. All aspects of this invention are particularly useful in postmenopausal women.

As may be seen from the scheme of FIG. 1, a number of sex hormones precursors released by the adrenals may be converted by a variety of non-adrenal biological pathways into estrogen in the peripheral tissues. Among the hormone precursors thus produced are 17β-estradiol and androst-5-ene-3β,17β-diol. It is therefore highly desirable to include an inhibitor of an enzyme that catalyzes a step in the synthesis of a sex steroid from such precursors. Such enzymes include, for example 17β-estradiol dehydrogenase or 17β-hydroxy steroid dehydrogenase. Such an inhibitor will close down the synthetic pathways crossed by vertical line 5 denoted “17β-HSD” on FIG. 1. Hence synthesis of both major forms of estrogen shown on FIG. 1 is substantially prevented. Other sex steroid formation inhibitors such as inhibitors of 3β-hydroxy steroid or of aromatase activity are also preferably included in treatment in order to close down the synthetic pathways crossed by the two horizontal lines 6 and 7 denoted “ARO” and “3β-HSD”, respectively. Aromatase is an enzyme that aids in aromatizing the A ring in the basic steroid structure.

It will be noted from FIG. 1 that the use of an aromatase inhibitor has the effect of reducing estrogen levels while potentially maintaining or even increasing androgen levels (androgen precursors are not converted into corresponding estrogens and therefore remain “available” as androgens). In contrast to the situation in which androgen secretion is also inhibited, use of an aromatase inhibitor (in addition to an antiestrogen) has the added benefit of potentially simplifying the regimen because there is no need to administer exogenous androgens. By choosing the combination of an antiestrogen with an appropriate enzyme inhibitor to minimize the adverse ADME interactions, an optimum composition can be obtained.

In summary then, the process for identifying a combination treatment of breast cancer in an animal comprises

identifying antiestrogen drugs and a therapeutically-effective dosage range for an antiestrogen so identified;

determining the relevant aspects of absorption, distribution, metabolism, and excretion characteristics of the antiestrogen for the animal;

identifying sex steroid enzyme inhibitor drug; and a therapeutically-effective dosage range for an enzyme inhibitor so identified;

determining the relevant aspects of absorption, distribution, metabolism, and excretion characteristics of an enzyme inhibitor so identified in the animal;

choosing each drug and a dosage range for each drug such that each drug exhibits useful therapeutic activity but exhibits minimal interference with the absorption, distribution, metabolism, and excretion of the other drug.

With our discovery of the manner of identifying a combination treatment of breast cancer, another aspect of the invention can be seen. This aspect of the invention is a method of treating breast cancer. The method is particularly useful in female patients with reduced hormonal secretions, e.g., postmenopausal women, women who have had their ovaries removed surgically, or women who have had their ovarian function suppressed by chemical means. The method comprises administering a therapeutically-effective amount of an antiestrogen agent and concurrently administering a therapeutically-effective amount of at least one other agent having tumor inhibitory activity, the agents and dosage ranges being chosen so that there is minimal material interference of one agent's ADME characteristics by the other agent. Preferably the other agent is a sex steroid enzyme inhibiting agent. Specifically, the other agent is an aromatase inhibiting agent.

Once the combination of these first two agents is determined, other agents can be included using the ADME analysis as described herein.

The other agents that can be included in the method of treatment include an androgen, a progestin or a glucocorticoid to fit into the appropriate receptor to aid the inhibition of the growth of the cancer. Other agents are inhibitors of growth hormone secretion, inhibitors of prolactin secretion, and inhibitors of adrenal corticotrophin hormone (ACTH) secretion. The latter has the effect of preventing ACTH from reaching the adrenals and thus of preventing the adrenals from synthesizing and secreting compounds such as DHEAS, a precursor in the synthesis of estrogen. Alternatively, inhibitors that close down synthetic pathways in the adrenals will achieve the same result. When adrenal secretions are inhibited or stopped, essential glucocorticoids should be added back as part of the therapy. However, all such additions much consider the requirements to effect, in aggregate, a NCE regimen, i.e., an optimized combination which minimizes the adverse interactions among the entities' ADME characteristics. The method of optimizing treatment or of treating breast cancer employs at least two agents and may include more.

In another aspect of the invention, i.e., the treatment of breast cancer in a human female whose ovarian function has been interrupted (naturally via menopause, by surgery or by other means), the method comprises administering to the woman a therapeutically effective amount of a properly selected antiestrogen and a properly selected other agent (e.g., one that inhibits an enzyme catalyzing sex steroid formation, such as an aromatase inhibitor) to provide an NCE regimen. In addition to the example given of an antiestrogen and the enzyme inhibitor in the treatment, at least one additional compound may be added, which includes an androgen, a progestin, a glucocorticoid, an inhibitor of prolactin secretion, an inhibitor of growth hormone, or an inhibitor of ACTH secretion. Mixtures of such compound are also useful.

In selecting candidate drugs in an NCE Regimen and determining how to judge the levels of components in the treatment regimen, one determines the effect of one component on the other's ADME, and optionally endocrine, profile. Candidates are evaluated and selected according to the hierarchy shown in Table I, with the fewer number and level of interferences being preferred.

The object of the evaluation of the effect of each drug on the ADME characteristics and optionally endocrine profile of the other is to avoid material interference between the two drugs. Material interference means that one drug is interfering with the ADME characteristics or endocrine function sufficiently to measurably reduce the efficacy of the other drug and/or measurably increase the toxicity of the other drug. One can avoid material interference by following the analysis set forth in the ensuing discussion. The analysis includes the endocrine profile as parts of the analysis. This is preferred but not absolutely required. Moreover, it may also be desirable to consider similarities or dissimilarities in chemical structures of molecules to be combined within a regimen and underlying pharmacokinetic parameters (including but not limited to times to maximal plasma level, absorption/distribution/elimination half-lives, route and extent of organ clearance, overall time to steady-state). The relevant aspects of absorption include factors such as the route of absorption (e.g., oral), the influence of timing, and the amount and type of food or caloric intake that might affect plasma levels. Relevant aspects of distribution include, i.a., the extent and characterization of plasma protein binding, while relevant aspects of excretion include the primary route(s) of elimination and the presence or absence of enterophepatic circulation. The relevant aspects of metabolism include the primary enzymes involved in metabolism or clearance, whether metabolizing enzymes for one drug are induced by another, and at what dose levels, the effect of other enzyme inducers, whether a metabolite is active and at what relative level, and the presence of significant pre-systemic metabolism. The relevant aspects of the endocrine effect includes the primary endocrine mechanism, whether feedback stimulation or inhibition occurs, and whether direction inhibition of a primary endocrine mechanism is operative. Other considerations include organ (e.g. liver or kidney) dysfunction influences on ADME parameters, demographic factors (e.g. as race, sex, age), genomic factors such as single nucleotide polymorphisms or haplotypes or allelic or chromosomal profiling. Others may be apparent to one of skill in the art.

As shown in Table II, there are five categories of possible interference, including interference with absorption, distribution, metabolism, excretion, or with endocrine function. Interferences may occur singly or in combination. It will be clear to those skilled in the arts that the following represent the number of possible combinations of interferences:

TABLE I
Number of Different Combinations of Interferences

| Number of | |
|---|---|
| Categories | Number of |
| with | Different |
| Interferences | Combinations |
| 0 | 1 |
| 1 | 5 |
| 2 | 10 |
| 3 | 10 |
| 4 | 5 |
| 5 | 1 |

In Table II are shown more detailed descriptions of the possible categories of interferences. In Table II, a drug is considered with respect to one other candidate drug in the regimen. This is a “first-order” ranking process. Smaller ranking numbers are, in general, preferable to larger ranking numbers because a smaller number indicate a smaller number of categories of interference without necessarily quantitating, however, the degree of interference for any one category.

TABLE II
Combinations of Interferences

| Number | | | | | | Interference | |
|---|---|---|---|---|---|---|---|
| of | | Interference | Interference | Interference | Interference | with | |
| Positive | Letter | with | with | with | with | Endocrine | Relative |
| Categories | Identifier | Absorption | Distribution | Metabolism | Excretion | Effect | Ranking |
| 0 | A | no | no | no | no | no | 1 |
| 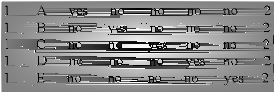 | | | | | | | |
| 2 | A | yes | yes | no | no | no | 3 |
| 2 | B | yes | no | yes | no | no | 3 |
| 2 | C | yes | no | no | yes | no | 3 |
| 2 | D | yes | no | no | no | yes | 3 |
| 2 | E | no | yes | yes | no | no | 3 |
| 2 | F | no | yes | no | yes | no | 3 |
| 2 | G | no | yes | no | no | yes | 3 |
| 2 | H | no | no | yes | yes | no | 3 |
| 2 | I | no | no | yes | no | yes | 3 |
| 2 | J | no | no | no | yes | yes | 3 |
| 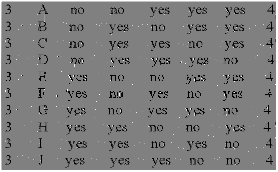 | | | | | | | |
| 4 | A | yes | yes | yes | yes | no | 5 |
| 4 | B | yes | yes | yes | no | yes | 5 |
| 4 | C | yes | yes | no | yes | yes | 5 |
| 4 | D | yes | yes | yes | no | yes | 5 |
| 4 | E | yes | yes | yes | yes | no | 5 |
| 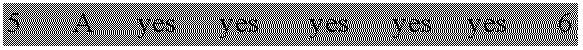 | | | | | | | |

The characteristics of the relative ranking of 1 are preferred for a single drug considered as part of a combination: There is no interference with important characteristics of other drugs. Accordingly, this is a good candidate molecule for an NCE regimen member. For a drug with a ranking of 6, with interferences in all categories, the drug would not be a good candidate molecule for an NCE regimen member.

The ranking process is repeated for each candidate drug. For two different drugs with the same first-order relative ranking (2, 3, 4 or 5) a second-order ranking process may be necessary.

For example, consider the five possibilities in Table III in which the number of positive categories is equal to 1 (each of the categories is also labeled with a letter, A-E, for clarity in comparisons within this category).

TABLE III
Example: One Positive Interference Category

| | | | | | | |
|---|---|---|---|---|---|---|
| | | | | | Interference | |
| Number of | Interference | Interference | Interference | Interference | with | |
| Positive | with | with | with | with | Endocrine | Relative |
| Categories | Absorption | Distribution | Metabolism | Excretion | Effect | Ranking |

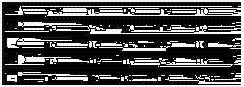

In each case there is an interference with one of the five categories. Interaction in the category 1-A may lead to reduced anti-tumor activity, which may not fully be correctable, even by giving higher doses of the drug in question because one drug is not absorbed due to the material interference by the other drug. Category 1-B can be associated with lower or higher bioavailability (due to improper distribution in the body) and, therefore, a higher or lower cost, respectively, to achieve a pharmaceutical effect. Whether lower or higher must be determined or estimated with reasonable accuracy. Category 1-B, interference with distribution, is best judged by plasma or tissue levels of active drug [or the active metabolite(s)] of the active drug. In category 1-C, if metabolism is accelerated, then 1-C is similar to 1-A (less parent drug is available), while if metabolism is retarded then 1-C is more similar to 1-D as described below. If the metabolite is the active moiety, however, then accelerated metabolism can also produce results more similar to those described in 1-D. Category 1-D is typically associated with higher bioavailability (i.e., less drug is excreted so more stays in the system) and, therefore, potentially it may be possible to administer less drug to obtain the same effect. Category 1-E, interference with an endocrine effect may be similar to 1-A, 1-B, or 1-C, where, for 1-C metabolism of the active moiety is accelerated.

In general, the goal will be to select the drug in the category that is associated with the administration of the smallest (and therefore most cost-effective) amount of drug to achieve a given therapeutic effect. An effect in a category does not always indicate the magnitude of the effect within that category, and quantitation is desirable where possible.

TABLE IV
Example: Two Positive Interference Categories

| Number | | | | | Interference | |
|---|---|---|---|---|---|---|
| of | Interference | Interference | Interference | Interference | with | |
| Positive | with | with | with | with | Endocrine | Relative |
| Categories | Absorption | Distribution | Metabolism | Excretion | Effect | Ranking |
| 2-A | yes | yes | no | no | no | 3 |
| 2-B | yes | No | yes | no | no | 3 |
| 2-C | yes | No | no | yes | no | 3 |
| 2-D | yes | No | no | no | yes | 3 |
| 2-E | no | yes | yes | no | no | 3 |
| 2-F | no | yes | no | yes | no | 3 |
| 2-G | no | yes | no | no | yes | 3 |
| 2-H | no | no | yes | yes | no | 3 |
| 2-I | no | no | yes | no | yes | 3 |
| 2-J | no | no | no | yes | yes | 3 |

Where two categories are affected by one drug, the analysis becomes more complex. In these scenarios, the interfering effects can be additive, synergistic, or antagonistic. For example, in 2-A, diminished absorption and diminished distribution can be adversely additive or synergistic (i.e., much less drug reaches the target sites). However, it is possible that a change in distribution can counterbalance a reduction in absorption. In 2-B and 2-C, diminished absorption may also possibly be countered by the interference with metabolism or excretion. In 2-D, the effects will most probably be adversely additive or synergistic (lower absorption and less endocrine effects will “add” to diminish the beneficial effect). In 2-E and 2-F, the effects may counteract one another although a change in distribution can add to the interference with metabolism under certain circumstances. In 2-G, the interference may be counteractive or may be additive or synergistic to diminish a drug effect, while in 2-H an additive or synergistic effect may actually enhance a drug effect. In 2-I and 2-J, effects may counteract one another. The complexity emphasizes the essential nature of the current invention to best choose drug combinations.

If each category from the Table II labeled “Combination of Interferences” is shown by number and letter, then each combination of interferences may be assigned a probable qualitative overall interaction value where the combination of interferences has an overall increase in or positive (POS) effect or an overall reduction in or negative (NEG) effect on the pharmacological activity of the drug that is the subject of the interaction. As noted, however, changes in pharmacological activity may also be accompanied by changes in toxicological activity. It is also possible that one interference may counteract, i.e. antagonize (ANT) the effect of another interference so that an overall POS effect or NEG effect becomes more difficult to predict without extensive and quantitative ADME data for both agents, singly and in combination. See Table V.

TABLE V
Probable Effect of Combinations of Interactions on a Target Drug

| | Probable Effect |
|---|---|
| Designation of | of Combination |
| Categories | of Inferences |
| 0-A | NONE |
| 1-A | NEG |
| 1-B | POS/NEG |
| 1-C | POS/NEG |
| 1-D | POS |
| 1-E | NEG |
| 2-A | ANT/NEG |
| 2-B | ANT/NEG |
| 2-C | ANT |
| 2-D | NEG |
| 2-E | POS/ANT/NEG |
| 2-F | POS/ANT |
| 2-G | ANT/NEG |
| 2-H | POS/ANT |
| 2-I | ANT/NEG |
| 2-J | ANT |
| 3-A | ANT |
| 3-B | ANT |
| 3-C | ANT |
| 3-D | ANT |
| 3-E | ANT |
| 3-F | ANT/NEG |
| 3-G | ANT |
| 3-H | NEG/ANT |
| 3-I | ANT |
| 3-J | ANT/NEG |
| 4-A | ANT |
| 4-B | ANT |
| 4-C | ANT |
| 4-D | ANT |
| 4-E | ANT |
| 5-A | ANT |

While the positive, negative, or antagonistic interactions described in the table above are qualitatively accurate, it is possible that one interference may so dominate the overall biological effects that the potential antagonism may be difficult to ascertain solely on a qualitative basis. As indicated, it is also possible that the overall interference effect of a category 1 or category 2 drug may be greater than the interference effect of a category 3, 4, or 5 drug, even though the latter categories have a larger “number” of interferences. In this instance, it is the net magnitude and direction of interference rather than the number of interferences that is more important. The complexity of these interactions, and the number of potentially interacting factors, serves further to underscore the deficiencies in prior art in the area of combination therapy in breast cancer.

When one drug has been subjected to such one or more drug-drug analyses as described above, then the next drug to be considered for an NCE regimen can be similarly analyzed. Then the relative benefits of one drug, considered in the framework of this NCE-regimen analysis, can be compared to those of the second drug, also having been considered in the same analytical framework. Relative risk assessments, relative efficacy assessments, and relative cost-of-goods assessment can be then performed and the most appropriate drug combination(s) selected.

The ADME and endocrine interferences between any two drugs can be determined by researching the information from literature or from database sources or by empirical means, i.e., from in vitro or in vivo experimentation.

Tools to consider employing to determine ADME interferences include pharmacokinetic and/or pharmacodynamic analyses in animals or in humans with standard single- or multi-compartment pharmacokinetic analyses of blood, plasma, tissue, or tumor levels. In vivo methods are explained in standard texts.

However, those skilled in the arts will recognize that plasma levels of estrogens (and sometimes plasma levels of drug(s)) are only surrogates for a final assessment of relative effectiveness and relative safety.

Once the qualitative and/or quantitative data are gathered for the combination of interest, the dosages are adjusted to eliminate or minimize the interference of one agent by another.

A practical example to demonstrate the application of the NCE algorithm may be seen in the concept of combining an anti-estrogen and an aromatase inhibitor for the first line therapy of advanced postmenopausal breast cancer. Since these patients are already postmenopausal, i.e., they have reduced ovarian hormonal secretions, a combination with an LH-RH agonist would not be seen as adding benefit to this combination. A recent publication by Santen et al (Yue W, Berstein L, Wang J P, Santen R J, Long term estrogen deprivation (LTED) enhances aromatase activity in breast cancer cells. Proceedings, 91st Annual Meeting of the American Association for Cancer Research, Volume 41, Abstract 2376, 2000) using an in vitro model of cultured estrogen-sensitive MCF-7 breast cancer cells in estrogen-deprived medium, demonstrated these cells to initially stop growing, but to consequently show comparable growth rate as in estrogen-containing medium. This model mimics growth inhibition by antiestrogens used clinically and suggests that estrogen-deprived cells remain responsive to estrogen but may require much lower amounts of estrogen for proliferation. Since the authors found aromatase activities in LTED cells to be 3-4 fold higher than in wild type MCF-7 cells, they conclude that LTED upregulates aromatase. Enhanced aromatase activity may be thus be partially responsible for the adaptation of breast cancer cells to low estrogen environment.

Consequently, the proposed combination of an antiestrogen blocking the estrogen receptor function and an aromatase inhibitor reducing the activity of tumoral aromatase is supported preclinically. Attempting to identify a most suitable combination of drugs, one would soon realize that there are currently two antiestrogens clinically available, tamoxifen and toremifene, as well as four aromatase inhibitors, including letrozole. Other representatives of each drug class are in clinical testing. Considering combinations of these compounds helps to exemplify the NCE regimen approach.

An agonistic effect of tamoxifen on the uterus was observed when it was given alone and when combined with an aromatase inhibitor, which indicates that the residual agonistic estrogenic signal caused by tamoxifen is both measurable and relevant and is not controlled by addition of aromatase inhibitors: (Brodie A, Lu Q, Liu Y, Long B, Wang J P, Yue W. Preclinical studies using the intratumoral aromatase model for postmenopausal breast cancer. Oncology (Hunting); 12(3 Suppl 5):36-40, 1998) If the antiestrogen in question were to be toremifene, the residual agnostic estrogenic signal at doses used clinically would be up to 40-fold less than that of tamoxifen as shown by Di Salle et al (Di Salle E, Zaccheo T, Ornati G, et al. Antiestrogenic and antitumor properties of the new triphenethylene derivative toremifene in the rat. Journal of Steroid Biochemistry. 36: 203-206, 1990).

Tamoxifen and letrozole represent one potential combination of an antiestrogen and aromatase inhibitor. However, it has been shown and published that estrogen suppression was not enhanced by this combination and, as a result, the antitumor effect of tamoxifen plus letrozole was less than expected. (Ingle J N, Suman V J, Johnson P A, Krook J E, Mailliard J A, Wheeler R H, Loprinzi C L, Perez E A, Jordan V C, Dowsett M. Evaluation of tamoxifen plus letrozole with assessment of pharmacokinetic interaction in postmenopausal women with metastatic breast cancer. Clin Cancer Res. July;5(7):1642-9, 1999).

In addition, during combination therapy with tamoxifen, plasma levels of letrozole could be reduced by a mean of 37% as compared to single agent treatment with letrozole alone, and this reduction persisted during months of combination therapy. (Dowsett M, Pfister C, Johnston S R et al. Impact of tamoxifen on the pharmacokinetics and endocrine effects of the aromatase inhibitor letrozole in postmenopausal women with breast cancer. Clin Cancer Res; 5(9):2338-43, 1999) Tamoxifen and letrozole share multiple cytochrome p450 isoenzymes, which have been shown to be induced by tamoxifen (Nuwaysir E, Dragan Y P, Jefcoate C R, et al. Effects of tamoxifen administration on the expression of xenobiotic metabolizing enzymes in the rat liver. Cancer Res; 55:1780-89, 1995). Combination therapy with tamoxifen can lead to increased metabolism. This combination would be adverse due to interference with metabolism. It is possible that this unexpected interaction of tamoxifen with another drug might affect the efficacy of other anticancer agents.

The above examples also make clear that information regarding possible interference does not necessarily have to rely on newly generated preclinical or clinical data, but may well be readily available from published references. It is necessary to utilize the methods described in the current invention, however, in order to make optimal use of such data. As part of the NCE approach a deliberate effort is made to collect, review and apply the available data for the improving treatment of patients with breast cancer.

In selecting component drugs for a NCE regimen it is also useful to consider and to balance the following characteristics of each drug being considered for inclusion: protein binding and the concentration or amount of excipients and activity, if any, of excipients.

In choosing the active agents useful in the various aspects of this invention, one should consider specific agents in the categories set forth hereinafter.

Antiestrogens

The antiestrogen compounds useful in the various aspects of this invention include many compounds that are well known in the art. Two preferred compounds are tamoxifen and toremifene, along with the pharmaceutically acceptable salts of these compound. Presently these compounds are commercially available as Novaldex® from Astra Zeneca Pharmaceuticals and Fareston® from Shire Pharmaceuticals.

Tamoxifen's chemical name is (Z)-2-[4-(1,2-diphenyl-1-butenyl)-phenoxyl]-N,N-dimethylethanamine or 1-p-β-dimethylaminoethoxyphenyl-trans-1,2-diphenylbut-1-ene. Its chemical formula is

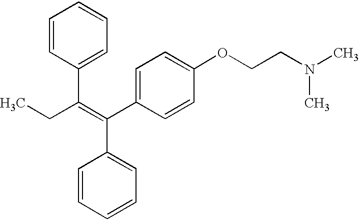

Toremifene's chemical name is (Z)-2-[4-(4-chloro-1,2-diphenyl-1-butenyl)phenoxy]-N,N-dimethylethanamine. Its chemical formula is

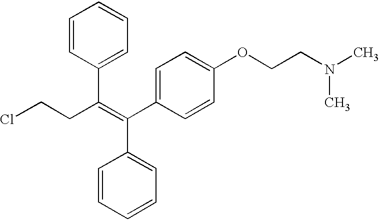

Others are set forth in U.S. Pat. No. 5,550,107 to Labrie, which is incorporated herein by reference. Typical suitable antiestrogens include those steroidal and non-steroidal antiestrogens such as (1 RS,2RS)-4,4′-diacetoxy-5,5′-difluoro-(1-ethyl-2-methylene)di-m-phenylenediacetate, which is available from Biorex under the trade name of Acefluranol; 6.alpha.-chloro-16.alpha.-methyl-pregn-4-ene-3,20-dione which is available from Eli Lilly & Co., Indianapolis, Ind. under the trade name of Clometherone; 6-chloro-17-hydroxypregna-1,4,6-triene-3,20-dione which is available as the acetate salt as Delmadione Acetate; 17-hydroxy-6-methyl-19-norpregna-4,6-diene-3,20-dione which is available from Theramex under the name of Lutenyl; 1-[2-[4-[1-(4-methoxyphenyl)-2-nitro-2-phenylethenyl)phenoxy]ethyl]-pyrrolidine which is available as the citrate salt from Parke-Davis Div. of Warner-Lambert Co., Morris Plains, N.J. under the name of Nitromifene Citrate; substituted aminoalkoxyphenylalkenes such as tamoxifen citrate salt from Stuart Pharmaceuticals, Wilmington, Del. (see also Belgian patent No. 637,389, Mar. 1964); 3,4-dihydro-2-(p-methoxyphenyl)-1-naphthyl-p-[2-(1-pyrrolidinyl)ethoxy]phenyl ketone which is available as the methane sulfonate salt from Eli Lilly & Co. under the tradename of Trioxifene Mesylate; 1-[4′-(2-phenyl)-bl-(3′-hydroxyphenyl)-2-phenyl-but-1-ene which is available from Klinge Pharma; [6-hydroxy-2-(p-hydroxyphenyl)-benzo(b)thien-3yl]-[2-(1-pyrrolidinyl)-ethoxy phenyl]ketone which is available from Eli Lilly & Co. (LY 117018); [6-hydroxy-2-(4-hydroxyphenyl)benzo(b)thien-3-yl]-[4-(2-(1-piperdinyl)ethoxy)phenyl]methanone, which is available from Eli Lilly & Co. as the hydrogen chloride salt (LY156758); meso-3,4-bis(3′-hydroxyphenyl) hexane as well as the dimethyl, dipropyl and 3′-acetoxy phenyl analogues which are described in U.S. Pat. No. 4,094,994; and a series of 1-phenyl-alkane and -alkenes, e.g.,(E)-3-cyclopentyl-1-(4-hydroxyphenyl)-1-phenyl-1-butene and 2-cyclo-pentyl-1-[4-hydroxy or methoxyphenyl]-3-phenyl-2-propen-1-ol and FC-1157 which are available as the citrate salt from Farmos Group, Ltd., Turku, Finland (see also Eur. Pat. Appln. Ep. No 78,158). It is preferred to use an antiestrogen which shows minimal partial estrogen agonism. Toremifene and tamoxifen are at the preferred antiestrogens of the class of those possessing some agonistic activity.

Other suitable antiestrogens also include 7.alpha.-substituents of estradiol (European Pat. No. 0138504) and non-steroidal compounds bearing a similar aliphalic side-chain (U.S. Pat. No. 4,732,912), both of which are incorporated herein by reference.

Other suitable antiestrogens may be found by accessing PHARMAPROJECTS data base. For example PHARMAPROJECTS accession number (PAN) 28929 refers to antiestrogen being developed by Schering Plough.

Other compounds include the following:

PAN 24611: The chemical name is 4-methyl-2[4-[2-(1-piperidinyl)ethoxy]phenyl]-7-(pivaloyloxy)-3-[4-(pivaloyloxy)phenyl]-2H-1-benzopyran (code name EM-800). The chemical formula is

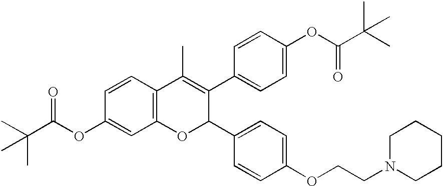

PAN28952: SH-646 being developed by Schering AG.

PAN18136: Lasofoxifene was originated by Ligand. The chemical name is 5,6,7,8-tetrahydro-6-phenyl-5(4-(2-(1-pyrrolidinyl)ethoxy)phenyl-(5R-cis)-2-naphthalenol, (S-(R*, R*))-2,3-dihydroxybutanedioate. The chemical formula is

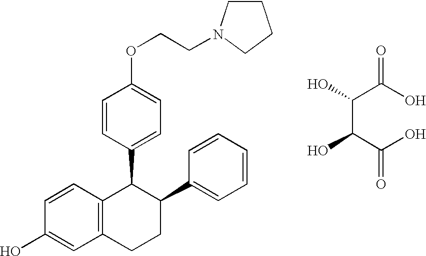

PAN25625: 2-methoxyestradiol is being developed by EntreMed. The chemical formula is

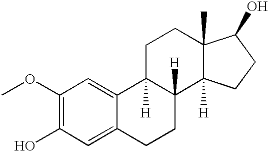

PAN25983: LY-326391 was originated at Eli Lilly. The chemical name is 2-(4-methoxyphenyl)-3-(4-(2-(1-piperidinyl)ethoxy)phenoxy)-benzo(b)thiophene-6-ol, hydrochloride. The chemical structure is

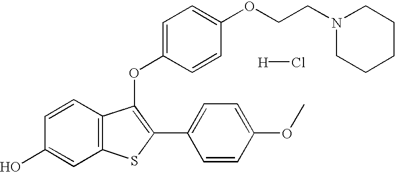

PAN13172: A-007 was originated by Dekk-Tec. The chemical name is 4,4′-dyhydroxybenzophenone-2,4-dinitrophenylhydrazone. The chemical structure is

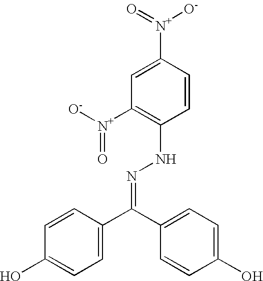

PAN28529: ERA 923 was originated by American Home Products and licensed to Ligand.

PAN15322: Fluvestront was originated by AstraZeneca. The chemical name is (7Alpha, 17β)-7-[9-[(4,4,5,5,5-pentafluoropentyl)sulfinyl]nonyl]-estra-1,3,5(10)-triene-3,17-diol. The chemical structure is

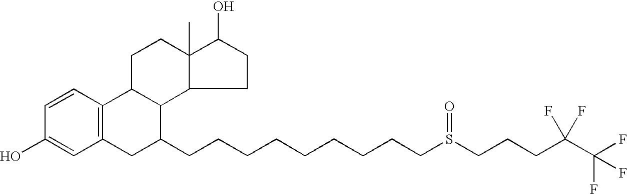

Enzyme Inhibitors

The sex steroid enzyme inhibiting drugs useful in the treatment regimen of this invention include many compounds that are well known in the art.

These agents can be viewed as inhibitors of sex steroid biosynthesis and are referred to herein as sex steroid enzyme inhibitors. Such compounds are those that inhibit biosynthesis of sex steroids from precursor steroids of adrenal and/or ovarian origin(s) preferably of both ovarian and adrenal origin. Their action is preferably exerted in the peripheral tissues, especially in the breast and the endometrium, and preferably inhibits aromatase activity. These latter compounds are aromatase inhibitors.

Inhibitors of sex steroid biosynthesis include but are not limited to (i) 3-(4-aminophenyl)-3-ethyl-2,6-piperidinedione which is commonly called aminoglutethimide, which is an inhibitor of sex steroid biosynthesis of adrenal but also of ovarian and testicular origin and which is available from Ciba Pharmaceutical Co., Summit N.J. under the trade name Cytadren, and (ii) ketoconazole, an effective testicular but also adrenal sex steroid biosynthesis, which is available from Janssen Pharmaceuticals, Piscataway, N.J., under the trade name Nizoral. Other inhibitors include 4-hydroxyandrostenedione and FCE 34304. Other exemplary compounds include atamestane, exemestane, anastrozole, fadrozole, finrozole, letrozole, vorozole, and YM511. The chemical names and structures of these compounds are as follows:

Atamestane: 1-methylandrosta-1,4-diene-3,17-dione.

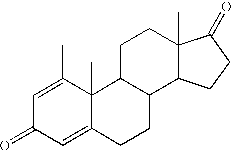

Exemestane: 6-Methylene androsta-1,4-diene-3,17-dione.

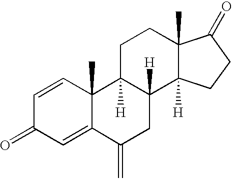

Anastrazole: Alpha,alpha,alpha,alpha′-tetramethyl-5-(1H-1,2,4-triazol-1-ylmethyl)-1,3-benzenediacetonitrile,

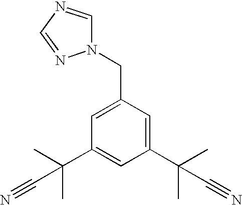

Fadrozole: 4-(5,6,7,8-Tetrahydroimidazo[1,5-a]pyridin-5-yl)-benzonitrile, monohydrochloride

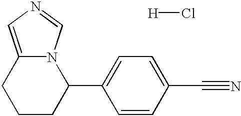

Finrozole: 4-(3-(4-Fluorophenyl)-2-hydroxy-1-(1H-1,2,4-triazol-1-yl)-propyl)-benzonitrile.

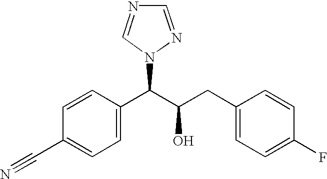

Letrozole:4,4′-(1H-1,2,4-triazol-1-ylmethylene)bis-benzonitrile

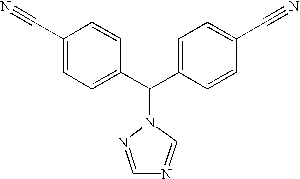

Vorozole: 6-[(4-chlorophenyl)-1H-1,2,4-triazol-1-ylmethyl]-1-methyl-1H-benzotriazole.

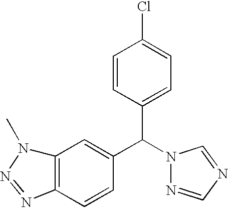

PAN 19816: YM-511 is being developed by Yamanouchi. The chemical name is: 4-[N-(4-bromobenzyl)-N-(4-cyanophenyl)amino]-4H-1,2,4-triazole.

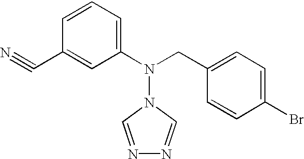

When an inhibitor of adrenal sex steroid biosynthesis, e.g., aminoglutethimide is administered, cortisol biosynthesis is blocked. Accordingly, a glucocorticoid, e.g., hydrocortisone, is preferably administered in physiological amounts sufficient to maintain normal glucocorticoid activity. Synthetic glucocorticoids, such as dexamethasone, can also be used.

Inhibitors of 3β-hydroxysteroid or .DELTA.5-.DELTA.4-isomerase activity, such as Trilostane, Eposlane or 4-MA, are also useful. Others, such as 16-methylene estrone and 16-methylene estradiol, act as specific inhibitors of 17β-estradiol dehydrogenase (Thomas et al., J. Biol. Chem. 258: 11500-11504, 1983).

Androgens

An androgen is a hormone that, among other activities, stimulates activity of the accessory male sex organs and encourages development of male sex characteristics. The androgenic agents useful in the treatment regimen of this invention include many compounds well known in the art.

Typically suitable androgens include 6-alpha-methyl,17-alpha-acetoxy progesterone or medroxyprogesterone acetate available, among others, from Upjohn and Farmitalia Carlo Erba, S.p.A. under the trade names, among others, of Provera and Farlutal, and the acronym MPA.

Other suitable androgens include certain compounds that can be described as synthetic progestins [see Labria et al. (Fertil. Steril. 31: 29-34, 1979)] and anabolic steroids (Raynaud and Ojasoo, Innovative Approaches in Drug Research. Elsevier Sc. Publishers, Amsterdam, pp. 47-72, 1986; Sandberg and Kirdoni, Pharmac. Ther. 36: 263-307, 1988; and Vincens, Simard and De Lignieres, Les Androgenes. In: Pharmacologie Clinique, Base de Therapeutique, 2i eme edition, Expansion Scientifique (Paris), pp. 2139-2158, 1988), anabolic steroids (Lamb, Am. 3. Sports Medicine 12, 31-38, 1984; Hilf, R. Anabolic-androgenic steroids and experimental tumors. In: (Kochachian, C. D., ed.), Handbook of Experimental Pharmacology, vol. 43, Anabolic-Androgenic Steroids, Springer-Verlag, Berlin, 725 pp., 1976). Examples of anabolic steroids include Calusterone (7β,17.alpha.-dimethyl-testosterone), fluoxymesterone (9.alpha.-fluoro-11β-hydroxy-17.alpha.-methyl testosterone), testosterone 17β-cyprionate, 17.alpha.-methyltestosterone, Pantestone (testosterone undecanoate), .DELTA.1-testololactone and Andractim. Some androgens also have progestin activity.

Progestins

A progestin is a substance that effects some or all of the changes produced by progesterone. Typically suitable progestins include 17,21-dimethyl-19-nor-4,9-pregnadiene-3,20-dione (“R5020, promegestone”) available from Roussel-UCLAF; cyproterone acetate (Androcur) available from Schering Ag.; 6-alpha-methyl, 17-alpha-acetoxy progesterone or medroxyprogesterone acetate (MPA) available from, among others, Upjohn and Farmitalia, Calbo Erba; Gestoden available from Shering; magestrol acetate (17.alpha.-acetoxy-6-methyl-pregna-4,6-diene-3,20-dione) available from Mead Johnson & Co., Evansville, Ind., under the trade name of Megace. Other progestins include Levolorgestrel, Gestodene, desogestrel, 3-keto-desogestrel, norethindrone, norethisterone, 13.alpha.-ethyl-17-hydroxy-18,19-dinor-17β-pregna-4,9,11-triene-20-yl-3-one (R2323), demegestone, norgestrienone, gastrinone, progesterone itself, and others described in Raynaud and Ojasoo, J. Steroid Biochem. 25: 811-833, 1986; Raynaud et al., J. Steroid Biochem. 12: 143-157, 1980; Raynaud, Ojasoo and Labrie, Steroid Hormones, Agonists and Antagonists, In: Mechanisms of Steroid Action (G. P. Lewis and M. Ginsburg, eds), McMillan Press, London, pp. 145-158 (1981).

Prolactin Secretion Inhibitors

A prolactin secretion inhibitor is an agent that reduces the production of the protein hormone prolactin from the pituitary gland. An example is bromocriptine available from Novartis as Parlodel.

Growth Hormone Secretion Inhibitors

A growth hormone secretion inhibitor is an agent that reduces the secretion of the protein growth hormone (also called somatotropin) from the pituitary gland. Examples include somatostatin (available from Novartis as Sandostatin), bromocriptine, and octreotide.

ACTH Secretion Inhibitors

An ACTH secretion inhibitor is an agent that reduces the secretion of the peptide hormone ACTH from the pituitary gland. An example is hydrocortisone, available as Solucortet from Pharmacia/Upjohn.

Reducing Ovarian Hormonal Secretions

As discussed previously, the various aspects of this invention are particularly useful for women in which the ovarian hormonal secretions are reduced. The invention is particularly valuable in postmenopausal women in whom the ovarian hormonal secretion is naturally reduced. Ovarian hormonal secretions also may be reduced by surgically removing the ovaries (oophorectomy) or chemically blocking secretion by administering an effective amount of an LHRH analog, which may be an LHRH agonist or antagonist. In one aspect, the present invention provides a method of treating breast cancer in a warm-blooded animal, which comprises administering (as part of a NCE regimen) to an animal in need of such treatment a properly selected LHRH analog, a properly selected antiestrogen, at least one properly selected inhibitor of sex steroid formation, and optionally a properly selected androgen, progestin, a glucocorticoid, or an inhibitor of prolactin secretion, growth hormone secretion, or ACTH secretion, in amounts sufficient to treat breast cancer.

While the LHRH analog may be an LHRH agonist or an LHRH antagonist, the use of a LHRH agonist is more preferred. Examples of LHRH analogs include leuprolide, nafarelin, goserelin, buserelin, and the like.

By the term “LHRH agonist” is meant synthetic analogues of the natural luteinizing hormone-releasing hormone (LHRH), for example, a decapeptide of the structure: L-pyroglutamyl-L-histidyl-L-tryptophyl-L-seryl-L-tyrosyl-glycyl-L-leucyl-L-arginyl-L-propylglycyl-NH2. Suitable LHRH agonists include nonapeptides and decapeptides represented by the formula: L-pyroglutamyl-L-histidyl-L-tryptophyl-L-seryl-L-tyrosyl-X-Y-arginyl-L-prolyl-Z wherein X is D-tryptophyl, D-leucyl, D-alanyl, iminobenzyl-D-histidyl, 3-(2-naphthyl)-D-alanyl, O-tert-butyl-D-seryl, D-tyrosyl, D-lysyl, D-phenylalanyl or N-methyl-D-alanyl and Y is L-leucyl, D-leucyl, N.alpha. -methyl D-leucyl, N.alpha. -methyl-L-leucyl or D-alanyl and wherein Z is glycyl-NHR1 or NHR1 wherein R1 is H, lower alkyl or lower haloalkyl. Lower alkyl includes straight- or branched-chain alkyls having 1 to 6 carbon atoms, e.g., methyl, ethyl, propyl, pentyl or hexyl, isobutyl, neopentyl and the like. Lower haloalkyl includes straight- and branched-chain alkyls of 1 to 6 carbon atoms having a halogen substituent, e.g., —CF3, —CH2 CF3, —CF2 CH3. Halogen means F, Cl, Br, I with Cl being preferred.

In preferred nonapeptides, Y is L-leucyl, X is an optically active D-form of tryptophan, serine (t-BuO), leucine, histidine (iminobenzyl), and alanine.

Preferred decapeptides include [D-Trp6 ]-LHRH wherein X-D-Trp, Y-L-leucyl, Z-glycyl-NH2, [D-Phe6]LHRH wherein X-D-phenylalanyl, Y-L-leucyl and Z-glycyl-HN3) or [D-Nal(2)6]LHRH which is [(3-(2-naphthyl)-D-Ala6]LH-RH wherein X-3-(2-naphthyl)-D-alanyl, Y-L-leucyl and Z-glycyl-NH3).

Other LHRH agonists useful within the scope of this invention are the .alpha.-aza analogues of the natural LH-RH, especially, [D-Phe6, Azgly10]-LHRH, [D-Tyr(Me)6, Azgly10]-LHRH, and [D-Ser-(t-BuO)6, Azgly10]-LHRH, disclosed by A. S. Dutta et al. in J. Med. Chem., 21, 1018 (1978) and U.S. Pat. No. 4,100,274 as well as those disclosed in U.S. Pat. Nos. 4,024,248 and 4,118,483.

Typical suitable LHRH antagonists include [N-Ac-D-p-Cl-Phe1,3,D-Phe3, D-Arg6, D-Ala10]LHRH disclosed by ]. Ercheggi et al., Biochem. Biophys. Res. Commun. 100, 915-920, (1981); [N-Ac-D-p-Cl-Phe1,2, D-Trp3, D-Arg6, D-Ala10]LHRH disclosed by D. H. Coy et al., Endocrinology, 110: 1445-1447, (1982); [N-Ac-D-(3-(2-naphthyl)-Ala)1, D-p-Cl-Phe2, D-Trp3, D-hArg(Et2)6, D-Ala10]-LHRH and [N-Ac-Prol, D-p-F-Phe2, (D-(3-(2-naphthyl)Ala3,6]-LHRH disclosed by J. J. Nestor et al. J. Steroid Biochem., 20 (No. 6B), 1366 (1984); the nona- and decapeptides analogs of LHRH useful as LHRH antagonists disclosed in U.S. Pat. No. 4,481,190 (J. J. Nestor et al.); analogs of the highly constrained cyclic antagonist, cycle [.DELTA.3 Prol, D-p-Cl-Phe2, D-Trp3,5, N-Me-Leu7,β-Ala10]LHRH disclosed by J. Rivier, ]. Steroid Biochem., 20, (No. 6B), 1365 (1984), and [N-Ac-D-(3-(2-naphthyl)-Ala1, D-p-F-Phe2, D-Trp3, D-Arg6]-LHRH disclosed by A. Corbin et al., J. Steroid Biochem. 20 (No. 6B) 1369 (1984).

Other LHRH agonist and antagonist analogs are disclosed in LHRH and its Analogues (B. H. Vickery et al. editors at page 3-10 (J. J. Nestor), 11-22 (J. Rivier et al.) and 23-33 (J. J. Nestor et al.).

The LHRH agonists and antagonists useful in this invention may conveniently be prepared by the method described by Stewart et al. in “Solid Phase Peptide Synthesis” (published in 1969 by Freeman & Co., San Francisco, page 1) but solution synthesis may also be used.

The nona- and decapeptides used in this invention are conveniently assembled on a solid resin support, such as 1% cross-linked Pro-Merrifield resin by use of an automatic peptide synthesizer. Typically, side-chain protecting groups, well known to those in the peptide arts, are used during the dicyclohexylcarbodiimide-catalyzed coupling of a tert-butyoxycarbonylamino acid to the growing peptide attached to a benzhydrylamine resin. The tert-butyloxycarbonyl protecting groups are removed at each stage with trifluoracetic acid. The nona- or decapeptide is cleaved from the resin and deprotected by use of HF. The crude peptide is purified by the usual techniques, e.g., gel filtration, HPLC and partition chromatography and optionally lyophilization. See also D. H. Coy et al., J. Med. Chem. 19, pages 423-452, (1976).

To reduce the ovarian hormonal secretions, a properly selected LHRH agonist is administered parenterally (e.g., subcutaneously, intranasally, intramuscularly) and a properly selected androgen, a properly selected antiestrogen, and at least one properly selected inhibitor of sex steroid formation are each administered orally.

In addition to the process for identifying a combination treatment and the method for treating breast cancer, other aspects of the invention relate to preventive aspects using the same ADME evaluation. Thus one aspect is a process for identifying a combination of drugs for prevention of breast cancer in an animal predisposed to such cancer. The process comprises

identifying antiestrogen drugs and a therapeutically-effective dosage range for an antiestrogen so identified,

determining the absorption, distribution, metabolism, and excretion characteristics for the antiestrogen,

identifying sex steroid enzyme inhibitor drugs and therapeutically-effective dosage ranges for an enzyme inhibitor so identified,

determining the absorption, distribution, metabolism, and excretion characteristics of an enzyme inhibitor so identified,

choosing each drug and a dosage range for each drug such that each drug exhibits useful therapeutic activity but each drug exhibits minimal interference with the absorption, distribution, metabolism, and excretion of the other drug.

Another aspect is a method of preventing breast cancer in an animal predisposed to such cancer. The process comprises

administering to the animal a therapeutically-effective amount of an antiestrogen drug and

concurrently administering a therapeutically-effective amount of a sex steroid enzyme inhibitor drug, wherein the antiestrogen and the enzyme inhibitor, and dosages for each, are chosen so that there is minimal material interference by one drug on the absorption, distribution, metabolism, and excretion of the other drug.

Still other aspects of the invention relate to a processes for optimizing treatment of a breast cancer patient or for optimizing a cancer-preventive regimen in an animal predisposed to such cancer. In either case, the process comprises

identifying antiestrogens and a therapeutically-effective dosage range for an antiestrogen so identified,

determining the absorption, distribution, metabolism, and excretion characteristics for the antiestrogen in the patient;

identifying sex steroid enzyme inhibitors and a therapeutically-effective dosage range for an enzyme inhibitor so identified;

determining the absorption, distribution, metabolism, and excretion characteristics for an enzyme inhibitor so identified;

selecting the antiestrogen and an enzyme inhibitor, and a dosage range for each, so that material interference is minimized with respect to the absorption, distribution, metabolism, and excretion characteristics of one towards the other; and

co-administering the selected antiestrogen and the enzyme inhibitor to the patient at the dosages selected.

The other agents discussed herein before (ie. an androgen, a progestin, a glucocorticoid, or an inhibitor of prolactin, ACTH, or growth hormone section) can be employed as discussed previously.

One further aspect of this invention relates to a kit that is useful in the method of treating or preventing breast cancer. The kit comprises an antiestrogen drug in a dosage form to provide a therapeutically-effective amount of the antiestrogen and a therapeutically-effective amount of a sex steroid enzyme inhibitor drug, wherein the dosage form and amount of each drug are chosen so that there is minimal material interference with respect to absorption, distribution, metabolism, and excretion characteristics of one drug towards the other drug. The kit may also include labeling instructions for the use of the combination to treat breast cancer in a patient or to prevent breast cancer in a patient that is predisposed to such cancer in accordance with the method of this invention. Other agents, as discussed hereinbefore, may be included in the kit in a dosage form and in an amount that do not interfere with the ADME characteristics among the active agents present.

A further aspect of this invention is a pharmaceutical composition for treating or preventing breast cancer that comprises a therapeutically-effective amount of an antiestrogen drug and a therapeutically-effective amount of a sex steroid enzyme inhibitor drug, wherein the amount of each drug is chosen so that there is minimal material interference between one drug's absorption, distribution, metabolism, and excretion characteristics and the other agent's absorption, distribution, metabolism, and excretion characteristics in a patient.

Thus, this invention provides a mechanism that leads to the effective treatment of breast cancer using an NCE regimen and provides teaching that is distinctly different than that from prior art wherein superficially similar combinations of drug classes can and have, in fact, led to suboptimal efficacy or excessive side effects.

By combining an optimal blockade of estrogen formation and/or action and the inhibitory effect of other agents on breast and endometrial cancer cell growth, the present invention provides a method of maximally inhibiting the growth of breast and endometrial cancer.

To assist in determining the effect of the treatment, plasma concentrations of the sex steroids of adrenal and ovarian origin, i.e., precursor steroids, androgens and estrogens, and tumor size may be measured. However, the proper selection of an NCE regimen, with predictably better outcomes, may be of more utility than use of the surrogate marker of plasma levels of estrogens. Lowered concentrations of sex steroids and reduction in tumor size may be indicative of successful treatment, e.g., inhibition of tumor growth using active compounds described herein in accordance with the present invention, although relatively raised levels could be expected if non-NCE regimens are employed. The concentrations of adrenal androgens and estrogens such as dehydroepiandrosterone (DHEA), DHEA-S sulfate (DHEAS), androst-5-ene-3β,17β-diol (.DELTA.′-diol) and, the ovarian estrogen, 17β-estradiol (E2) are measured by standard methods well known to those skilled in the art, see for example, F. Labrie et al., The Prostate 4, 579-584, 1983; Luthy et al., J. Gynecol. Endocrinol., 1, 151-158, 1987).

The change in tumor size is measured by standard physical methods well known to those skilled in the art, e.g., bone scan, chest X-ray, skeletal survey, ultrasonography, nuclear medicine scans, computerized tomography, magnetic resonance imaging, positron emission tomography, physical examination, and the like.

The following example provides a representative combination that is useful in the method, process, kit, and composition aspects of the invention.

### EXAMPLE I

### Application of the Process to Atamestane and Toremifene*

An example of the application of the method is shown in the table. The chemical structures, pharmacokinetic profiles, and the patterns of the absorption, distribution, metabolism, and excretion have little or no overlap in the categories an related subcategories. This lack of overlap suggests little or no possible adverse drug-drug interaction and, therefore, that atamestane and toremifene are an example of an NCE regimen.

| Factors to be considered in | | |
|---|---|---|
| the application of the process | Atamestane | Toremifene |
| Chemical structure (See “Detailed | steroid | non-steroid |
| Description”) | | |
| Pharmacokinetic profile | | |
| tₘₐₓ (time) | 1 hour | 3 hours |
| distribution t_{1/2} (time) | <1 hour | 4 hours |
| elimination t_{1/2} (time) | <24 hours | 5 days |
| total clearance (volume/unit time) | 84 liters/hour | 5 liters/hour |
| overall time to steady state | hours | 4-6 weeks |
| Absorption | | |
| route | oral | oral |
| influenced by food | high fat diet | no |
| | increases plasma | |
| | levels | |
| Distribution | | |
| plasma protein binding (%) | 80-90 | >99.5 |
| principal plasma binding protein(s) | a-1 acid glycoprotein; | albumin |
| | steroid binding | |
| | globulin(s); albumin | |
| Excretion | | |
| primary route of elimination | renal | fecal |
| enterohepatic circulation | no | yes |
| Metabolism | | |
| primary enzyme(s)* | 5β reductase; | CYP3A4 |
| | 17β hydroxysteroid- | |
| | dehyrogenase; mixed | |
| | function oxidase | |
| induction of primary metabolizing enzyme | no | no |
| plasma levels affected by known inducers of | no | yes |
| cytochrome p-450 enzyme system | (no change in | (2-fold increase in |
| | pharmacokinetics) | clearance and |
| | | decrease in half-life) |
| primary metabolite active | yes | yes |
| significant pre-systemic metabolism | yes | no |
| Endocrine effect | | |
| primary mechanism | enzyme inhibition | receptor blockade |
| feedback stimulation on combination drug’s | no | yes |
| primary mechanism | | |
| direct inhibition of primary mechanism by | no | no |
| combination drug | | |
| estrogen receptor binding with residual | no | yes |
| estrogenic effect | | |
| Other Effects on ADME or PK | | |
| age of patient | no | yes: increases in |
| | | elimination t_{1/2} and |
| | | volume of distribution |
| | | with increasing age |
| sex of patient | no | no known effect |
| race of patient | unknown | unexpected |
| decreases in renal function | unexpected | not applicable |
| decreases in hepatic function | unexpected | yes: increases in |
| | | elimination t_{1/2} and |
| | | decreasing function |
| *Using standard methodology (adapted from Tucker et al., 2001, “Optimizing Drug Development: Strategies to Assess Drug Metabolism/Transporter Interaction Potential Pharmaceutical Research, “Pharmaceutical Research 18: 1071-80; see also Tucker et al., 2001, “Optimizing Drug Development: Strategies to assess Metabolism/Transporter Interaction Potential--Towards a Consensus,” Br. J. Clin. Pharmacol. 52(1):107-117; Tucker et al., 2001, “EUFEPS Conference Report. | | |
| # Optimizing Drug Development: Strategies to assess Metabolism/Transporter Interaction Potential--Towards a Consensus,” Eur. J. Pharm. Sci. 13(4):417-428; Tucker et al., 2001, “Optimizing Drug Development: Strategies to assess Metabolism/Transporter Interaction Potential--Towards a Consensus,” Clin. Pharmacol. Ther. 70(2):103-114), we have demonstrated that the combination of atamestane and toremifene (0.1 and 5.0 μg/mL, respectively) does not substantially inhibit CYP isoenzymes | | |
| # 1A2, 3A4, 2C19, 2D6 or 2E1. | | |

By reviewing the ADME characteristics in the above table one can see that there is minimal material interference between atamestane (an aromatase inhibitor) and toremifene (an antiestrogen). With regard to absorption, both drugs are orally absorbed and only atamestane is influenced by food, i.e. plasma levels are increased by a high fat diet. Regarding distribution, both bind to plasma protein, but each binds to a different protein. Each drug is primarily excreted by a different route, and while toremifene is subject to enterohepatic circulation, atamestane is not. Each drug's primary enzymatic metabolism differs as shown and the other factors establish that there should be little if any interference. With regard to the endocrine effect, the primary mechanism for atamestane is enzyme inhibition, while that of toremifene is receptor blockade.

A useful regimen for this combination is to administer toremifene once a day orally at a standard dose and administer 500 mg of atamestane daily, preferably 300 mg in the morning and 200 mg in the evening about 12 hours later.

One skilled in the art will also recognize that a properly chosen NCE regimen will also be useful in the treatment or prevention of other types of cancer such as endometrial cancer. One skilled in the art will also recognize that one or more aspects of this invention must be used to optimally treat or prevent a disorder (particularly metastatic breast cancer in postmenopausal women) using a rational combination of appropriate drugs.

The terms and descriptions used herein are preferred embodiments set forth by way of illustration only and are not intended as limitations on the many variations which those of skill in the art will recognize to be possible in practicing the present invention as defined by the following claims.

The subject matter claimed is:

1. A process for identifying a combination of drugs for treatment of breast cancer in an animal, which process comprises
identifying antiestrogen drugs and a therapeutically-effective dosage range for an antiestrogen so identified,
determining the relevant aspects of absorption, distribution, metabolism, and excretion characteristics for the antiestrogen,
identifying sex steroid enzyme inhibitor drugs and therapeutically-effective dosage ranges for an enzyme inhibitor so identified,
determining the relevant aspects of absorption, distribution, metabolism, and excretion characteristics of an enzyme inhibitor so identified,
choosing each drug and a dosage range for each drug such that each drug exhibits useful therapeutic activity but each drug exhibits minimal interference with the absorption, distribution, metabolism, and excretion of the other drug.

2. The process of claim 1, wherein the animal is a human female.

3. The process of claim 2, wherein the human female exhibits reduced ovarian hormonal secretions.

4. The process of claim 3, wherein the human female is postmenopausal.

5. The process of claim 2, wherein the combination treatment is for a human female previously untreated by chemical means for the breast cancer.

6. The process of claim 2, wherein the combination treatment is for a human female previously treated for breast cancer through administration of an antiestrogen alone or an enzyme inhibitor alone.

7. The process of claim 1, wherein the antiestrogen is tamoxifen, toremifene, or EM-800.

8. The process of claim 1, wherein the enzyme inhibitor is an aromatase inhibitor.

9. The process of claim 8, wherein the aromatase inhibitor is atamestane and the antiestrogen is toremifene.

10. A method of treating breast cancer in an animal, which method comprises
administering to the animal a therapeutically-effective amount of an antiestrogen drug and
concurrently administering a therapeutically-effective amount of a sex steroid enzyme inhibitor drug, wherein the antiestrogen and the enzyme inhibitor, and dosage ranges for each, are chosen so that there is minimal material interference by one drug on the absorption, distribution, metabolism, and excretion (ADME) of the other drug.

11. The method of claim 10, wherein the animal is a human female.

12. The method of claim 11, wherein the human female exhibits reduced ovarian hormonal secretions.

13. The method of claim 12, wherein the human female is postmenopausal.

14. The method of claim 11, wherein the human female has not been previously treated by chemical means for the breast cancer.

15. The method of claim 11, wherein the human female was previously treated for breast cancer through administration of an antiestrogen alone or an enzyme inhibitor alone.

16. The method of claim 10, wherein the antiestrogen is tamoxifen, toremifene, or EM-800.

17. The method of claim 10, wherein the enzyme inhibitor is an aromatase inhibitor.

18. The method of claim 17, wherein the aromatase inhibitor is atamestane.

19. The method of claim 18, wherein the antiestrogen is toremifene.

20. A process for identifying a combination of drugs for prevention of breast cancer in an animal predisposed to such cancer, which process comprises
identifying antiestrogen drugs and a therapeutically-effective dosage range for an antiestrogen so identified,
determining the relevant aspects of absorption, distribution, metabolism, and excretion characteristics for the antiestrogen,
identifying sex steroid enzyme inhibitor drugs and therapeutically-effective dosage ranges for an enzyme inhibitor so identified,
determining the relevant aspects of absorption, distribution, metabolism, and excretion characteristics of an enzyme inhibitor so identified,
choosing each drug and a dosage range for each drug such that each drug exhibits useful therapeutic activity but each drug exhibits minimal interference with the absorption, distribution, metabolism, and excretion of the other drug.

21. The process of claim 20, wherein the animal is a human female.

22. The process of claim 21, wherein the human female exhibits reduced ovarian hormonal secretions.

23. The process of claim 22, wherein the human female is postmenopausal.

24. The process of claim 21, wherein the combination is for a human female previously untreated by chemical means for the breast cancer.

25. The process of claim 21, wherein the human female was previously treated for breast cancer through administration of an antiestrogen alone or an enzyme inhibitor alone.

26. The process of claim 20, wherein the antiestrogen is tamoxifen, toremifene, or EM-800.

27. The process of claim 20, wherein the enzyme inhibitor is an aromatase inhibitor.

28. The process of claim 27, wherein the aromatase inhibitor is atamestane.

29. The process of claim 28, wherein the antiestrogen is toremifene.

30. A method of preventing breast cancer in an animal predisposed to such cancer, which process comprises
administering to the animal a therapeutically-effective amount of an antiestrogen drug and
concurrently administering a therapeutically-effective amount of a sex steroid enzyme inhibitor drug, wherein the antiestrogen and the enzyme inhibitor, and dosages for each, are chosen so that there is minimal material interference by one drug on the absorption, distribution, metabolism, and excretion of the other drug.

31. The method of claim 30, wherein the animal is a human female.

32. The method of claim 31, wherein the human female exhibits reduced ovarian hormonal secretions.

33. The method of claim 32, wherein the human female is postmenopausal.

34. The process of claim 31, wherein the combination is for a human female previously untreated by chemical means for the breast cancer.

35. The process of claim 31, wherein the combination is for a human female previously treated for breast cancer through administration of an antiestrogen alone or an enzyme inhibitor alone.

36. The method of claim 30, wherein the antiestrogen is tamoxifen, toremifene, or EM-800.

37. The method of claim 30, wherein the enzyme inhibitor is an aromatase inhibitor.

38. The method of claim 36, wherein the aromatase inhibitor is atamestane.

39. The method of claim 37, wherein the antiestrogen is toremifene.

40. A process for optimizing treatment of a breast cancer patient, which process comprises
identifying antiestrogens and a therapeutically-effective dosage range for an antiestrogen so identified,
determining the relevant aspects of absorption, distribution, metabolism, and excretion characteristics for the antiestrogen in the patient;
identifying sex steroid enzyme inhibitors and a therapeutically-effective dosage range for an enzyme inhibitor so identified;
determining the relevant aspects of absorption, distribution, metabolism, and excretion characteristics for the enzyme inhibitors so identified;
selecting the antiestrogen and an enzyme inhibitor, and a dosage range for each, so that material interference is minimized with respect to the absorption, distribution, metabolism, and excretion characteristics of one towards the other; and
co-administering the selected antiestrogen and the enzyme inhibitor to the patient at the dosages selected.

41. The process of claim 40, wherein the animal is a human female.

42. The process of claim 41, wherein the human female exhibits reduced ovarian hormonal secretions.

43. The process of claim 42, wherein the human female is postmenopausal.

44. The process of claim 40, wherein the optimized treatment is for a patient previously untreated by chemical means for the breast cancer.

45. The process of claim 40, wherein the optimized treatment is for patient previously treated for breast cancer through administration of an antiestrogen alone or an enzyme in inhibitor alone.

46. The process of claim 40, wherein the antiestrogen is tamoxifen, toremifene, or EM-800.

47. The process of claim 40, wherein the enzyme inhibitor is an aromatase inhibitor.

48. The process of claim 47, wherein the aromatase inhibitor is atamestane.

49. The process of claim 48, wherein the antiestrogen is toremifene.

50. A process for optimizing a cancer-preventive regimen in an animal predisposed to such cancer, which process comprises
identifying antiestrogens and a therapeutically-effective dosage range for an antiestrogen so identified;
determining the relevant aspects of absorption, distribution, metabolism, and excretion characteristics for the antiestrogen in the animal;
identifying sex steroid enzyme inhibitors and a therapeutically-effective dosage range for an enzyme inhibitor so identified;
determining the relevant aspects of absorption, distribution, metabolism, and excretion characteristics for an enzyme inhibitor so identified;
selecting the antiestrogen and an enzyme inhibitor, and a dosage range for each, so that material interference is minimized with respect to the absorption, distribution, metabolism, and excretion characteristics of one towards the other; and
co-administering the chosen antiestrogen and the enzyme inhibitor to the animal at the dosages selected.

51. The method of claim 50, wherein the animal is a human female.

52. The method of claim 51, wherein the human female exhibits reduced ovarian hormonal secretions.

53. The method of claim 52, wherein the human female is postmenopausal.

54. The method of claim 51, wherein the female has not been previously treated by chemical means for the breast cancer.

55. The method of claim 51, wherein the female was previously treated for breast cancer through administration of an antiestrogen alone or an enzyme or inhibitor alone.

56. The method of claim 51, wherein the antiestrogen is tamoxifen, toremifene, or EM-800.

57. The method of claim 50, wherein the enzyme inhibitor is an aromatase inhibitor.

58. The method of claim 57, wherein the aromatase inhibitor is atamestane.

59. The method of claim 58, wherein the antiestrogen is toremifene.

60. The process of claim 1, 20, 40 or 50, which further comprises
identifying a further agent chosen from androgens, progestins, glucocorticoids, prolactin secretion inhibitors, growth hormone secretion inhibitors, and ACTH secretion inhibitors;
determining the relevant aspects of ADME characteristics of the further agent in the animal;
choosing the further agent and a therapeutically-effective dosage range therefor to combine with the antiestrogen and the enzyme inhibitor in a manner that minimizes material interference of the ADME characteristics between each agent.

61. The method of claims 10 or 30, which further comprises
concurrently administering a therapeutically-effective amount of a further agent chosen from androgens, progestins, glucocorticoids, prolactin secretion inhibitors, growth hormone secretion inhibitors, and ACTH secretion inhibitors; wherein the further agent and its dosage range is chosen to minimize material interference of the ADME characteristics between each agent.

62. A kit useful for treating breast cancer in a patient in need of treatment, which kit comprises
an antiestrogen drug in a dosage form to provide a therapeutically-effective amount of the antiestrogen and
a therapeutically-effective amount of a sex steroid enzyme inhibitor drug, wherein the dosage form and amount of each drug are chosen so that there is minimal material interference with respect to absorption, distribution, metabolism, and excretion characteristics of one drug towards the other drug.

63. The kit of claim 62, wherein the antiestrogen is tamoxifen, toremifene, or EM-800.

64. The kit of claim 62, wherein the enzyme inhibitor is an aromatase inhibitor.

65. The kit of claim 64, wherein the aromatase inhibitor is atamestane.

66. The kit of claim 65, wherein the antiestrogen is toremifene.

67. The kit of claim 62 that further comprises a therapeutically effective amount of another drug chosen so that the amount administered has minimal material interference on the absorption, distribution, metabolism, and excretion characteristics of the antiestrogen and enzyme inhibitor, and the antiestrogen and enzyme inhibitor have minimal material interference with the absorption, distribution, metabolism, and excretion characteristics of the other drug, wherein the other drug is an androgen, a progestin, a glucocorticoid, a prolactin secretion inhibitor, an ACTH secretion inhibitor, or a growth hormone secretion inhibitor.

68. A kit useful for preventing breast cancer in a patient predisposed to such cancer, which kit comprises
an antiestrogen drug in a dosage form to provide a therapeutically effective amount of the antiestrogen and
a therapeutically effective amount of an sex steroid enzyme inhibitor drug, wherein the dosage form and amount of each drug are chosen so that material interference is minimized with respect to absorption, distribution, metabolism, and excretion characteristics of one drug towards the other drug.

69. The kit of claim 68, wherein the antiestrogen is tamoxifen, toremifene, or EM-800.

70. The kit of claim 68, wherein the enzyme inhibitor is an aromatase inhibitor.

71. The kit of claim 70, wherein the aromatase inhibitor is atamestane.

72. The kit of claim 71, wherein the antiestrogen is toremifene.

73. The kit of claim 62 that further comprises a therapeutically effective amount of another drug chosen so that the amount administered has minimal material interference on the ADME characteristics of the antiestrogen and enzyme inhibitor, and the antiestrogen and enzyme inhibitor have minimal material interference with the absorption, distribution, metabolism, and excretion characteristics of the other agent, wherein the other agent is an androgen, a progestin, a glucocorticoid, a prolactin secretion inhibitor, an ACTH secretion inhibitor, or a growth hormone secretion inhibitor.

74. A pharmaceutical composition for treating or preventing breast cancer that comprises a therapeutically-effective amount of an antiestrogen drug and a therapeutically-effective amount of a sex steroid enzyme inhibitor drug, wherein the amount of each drug is chosen so that there is minimal material interference between one drug's absorption, distribution, metabolism, and excretion characteristics and the other agent's ADME characteristics in a patient.

75. The composition of claim 74, wherein the enzyme inhibitor is an aromatase inhibitor.

76. The composition of claim 75, wherein the aromatase inhibitor is chosen from atamestane, exemestane, anastrozole, fadrozole, findrozole, letrozole, vorozole, or YM-511.

77. The composition of claim 76, wherein the aromatase inhibitor is atamestane.

78. The composition of claim 75, wherein the antiestrogen is tamoxifen, toremifene or EM-800.

79. The composition of claim 77, wherein the antiestrogen is toremifene.
